(19) United States
(12) Patent Application Publication
(10) Pub. No.: US 2024/0130616 A1
(43) Pub. Date: Apr. 25, 2024

(54) TOUCH-FREE INFECTIOUS DISEASE SCREENING

(71) Applicant: KONINKLIJKE PHILIPS N.V., Eindhoven (NL)

(72) Inventors: Cornelis van Zon, Everett, MA (US); Joseph James Frassica, Gloucester, MA (US); Ali Akbar Ahmad Samadani, Somerville, MA (US); Wee Kar Tan, Medford, MA (US); Haibo Wang, Melrose, MA (US)

(21) Appl. No.: 18/275,235

(22) PCT Filed: Feb. 2, 2022
(86) PCT No.: PCT/EP2022/052410

§ 371 Date: Jul. 31, 2023

Related U.S. Application Data
(60) Provisional application No. 63/145,730, filed on Feb. 4, 2021.

(51) Int. Cl. A61B 5/00; A61B 5/15; G16H 40/67; G16H 50/20

(52) U.S. Cl. CPC A61B 5/0017; A61B 5/150786; G16H 40/67; G16H 50/20

(57) ABSTRACT

A security gateway for touch-free infectious disease screening includes a contactless sensor, a memory and a processor. The contactless sensor is configured to obtain health readings of a subject. The memory stores instructions. The processor executes the instructions. When executed by the processor, the instructions cause the security gateway to measure vital signs based on the health readings of the subject, and determine whether to allow access to a premise based on the vital signs.

### CROSS-REFERENCE TO RELATED APPLICATION

The present application claims priority under 35 U.S.C. § 119(e) and under 37 C.F.R. § 1.78(a) to commonly owned U.S. Provisional Application No. 63/145,730 filed on Feb. 4, 2021. The entire disclosure of U.S. Provisional Application No. 63/145,730 is specifically incorporated herein by reference in its entirety. (A copy of U.S. Provisional Application No. 63/145,730 is attached.)

### BACKGROUND

### Technical Field

The present disclosure relates to a device, system, method, and computer-readable medium for infectious disease screening. In particular, the present disclosure relates to a set of relatable solutions for screening of users for infectious disease employing contactless measurement of multiple vital signs.

### Description of the Related Art

During an outbreak of an infectious disease, the spread of a pathogen from human to human may be proactively combatted through various, often complementary, means. One such solution for combatting infectious disease may be containment. Containment may comprise utilization of measures to prevent the pathogen from spreading to new parts of a population. If successful, containment may result in preventing a pathogen from spreading to new hosts, potentially stopping spreading once all infected individuals are no longer contagious. Additionally, complementary measures such as vaccination or application of a cure may also be used to combat the spread of a pathogen, and these three mechanisms may often be used simultaneously.

Immediately after the outbreak of a disease, a vaccination or cure for the disease may not exist or be widely available. Further, in the case of a novel disease, development and implementation of a vaccine may take a year or longer. In the interim, containment may be the only mechanism by which the spread of a disease can be limited.

There are various challenges that may be associated with containment and implementation of containment measures. These may include, but are not limited to, unawareness of being infected, individuals ignoring medical advice, individuals ignoring quarantine orders, and others.

Unawareness of being infected may limit the effectiveness of containment measures. The symptoms of a disease may not appear, or may not be readily noticeable by an infected individual or others, until sometime after infection. The period of time between getting infected and symptoms appearing is called the incubation period. As an example, for COVID-19 this incubation period may be 2-14 days. During the incubation period, individuals may be infected, and may also be contagious, but may not realize that they are infected by the disease. These individuals may thereby spread the disease to others without knowledge of doing so. Further, individuals exhibiting symptoms may not realize those symptoms are caused by an infectious disease. Additionally, individuals may be infected and contagious, but never show symptoms of a disease. These scenarios and others may limit the effectiveness of containment measures by resulting in circumstances where an individual is contagious and may spread the disease without the individual knowing of his or her in-fact infection, as individuals who are unaware of having an infectious disease may be less likely to take precautions against spreading the disease.

Individuals may ignore medical advice, which may also limit the effectiveness of containment measures. Individuals who test positive may knowingly ignore voluntary quarantine advice. Individuals who may not have been tested but have an increased risk of infection due to, e.g., recently visiting a high-risk area, recently being in close proximity to infected people, or exhibiting symptoms indicative of infectious disease. These scenarios and others may limit the effectiveness of containment measures by resulting in spread by individuals who may be knowingly infected or at relatively high likelihood for infection.

Individuals may ignore quarantine orders, which may also limit the effectiveness of containment measures. Similar to individuals ignoring medical advice, individuals ignoring quarantine orders may limit the effectiveness of containment measures resulting in spread by individuals who may be knowingly infected or at relatively high likelihood for infection.

Some approaches exist to attempt to mitigate the effects of the above-mentioned challenges to effectiveness of containment measures. One such approach is screening. Screening may include active assessment of individuals seeking to enter an area, such as, e.g., a crowded or otherwise sensitive area, such as office buildings, airports, train stations, theaters, stadiums, etc. The goal of screening may generally be to prevent or reduce the likelihood of individuals with high likelihood of infection from entering the area by denying those individuals entry.

One example method of screening for entry into an area may be detection of fever through temperature measurement. Fever is often a symptom of infectious disease, and so assessment of individuals for fever, via, e.g., hand-held infrared thermometers or thermal cameras, may be used to identify individuals with a fever and subsequently reject those individuals from entry into the area. This method of screening may also be used on a large scale, such as through thermal cameras identifying individuals in a crowd as having a fever and enabling their removal from the crowd. These methods of screening are only examples, and other methods exist.

In areas where a high throughput of individuals is desired, commonly used screening methods, such as with infrared thermometers or thermal cameras, when used to screen individuals for entry to that area, may preclude throughput at a desired or acceptable level. This may occur due to, e.g., many individuals seeking to enter the area, the time required to screen each individual, or a combination of these two considerations.

Screening may contribute to disease spread, such as via interaction between an individual being screened and an individual taking vitals without proper protective equipment. Any people-facing aspects of screening may run this risk if the screening is done in a way that requires proximity of the individual(s) being screened to an individual or individuals who conduct the screening.

Inherently, screening of any type can result in false positives and false negatives. In an example context, a false positive may occur when screening for individuals exhibiting symptoms of COVID-19, and identifying an individual who has the flu, but not COVID-19, due to similarities between the symptoms of each disease, as having COVID-19. In another exemplary context, a false negative may occur when screening for individuals exhibiting symptoms of COVID-19 and failing to identify an individual who has the disease. Of these two risks, false negatives may be more dangerous because they allow infected individuals to enter a premise.

False negatives may result from a number of factors. These factors may include, but are not limited to, incubation period of a disease, where individuals are infected but do not show symptoms; the variability of symptoms across individuals, as different individuals may exhibit differing symptoms, differing severity of symptoms, or no symptoms at all; the waxing and waning of a fever, making an individual alternate between hyperthermic and hypothermic conditions as his or her body fights the disease; and others.

A need exists, therefore, for devices, systems, methods, and/or computer-readable mediums that provide an approach for screening that is more comprehensive than many commonly used screening measures, by achieving higher sensitivity and specificity while maintaining safety and an acceptable screening time, and that may be contactless.

### SUMMARY

According to an aspect of the present disclosure, a communication device for touch-free infectious disease screening includes a memory, a contactless sensor, a user interface, and a processor. The memory stores instructions. The contactless sensor is configured to obtain health readings of a subject. The user interface is configured to interactively obtain information from the subject in response to prompts. The processor executes the instructions. When executed by the processor, the instructions cause the communication device to measure vital signs based on the health readings of the subject; control the user interface to present the prompts, and interpret the information interactively obtained from the subject in response to the prompts; and encode results of measuring the vital signs and interpreting the information in a two-dimensional visualizable code. Access to a premise is conditioned on the two-dimensional visualizable code.

According to another aspect of the present disclosure, a method for touch-free infectious disease screening includes storing first instructions in a first memory of a communication device; obtaining first health readings of a subject via a first contactless sensor of the communication device; interactively obtaining first information from the subject in response to first prompts via a first user interface of the communication device; and executing the first instructions by a first processor of the communication device. When executed by the first processor, the first instructions cause the communication device to: measure first vital signs based on the first health readings of the subject; control the first user interface of the communication device to present the first prompts, and interpret the first information interactively obtained from the subject in response to the first prompts; and encode first results of measuring the first vital signs and of interpreting the first information in a two-dimensional visualizable code. The method also includes storing second instructions in a second memory of a security gateway; and executing the second instructions by a second processor of the security gateway. When executed by the second processor of the security gateway, the second instructions cause the security gateway to: scan the first user interface of the communication device to obtain the two-dimensional visualizable code;

decode the two-dimensional visualizable code; determine whether the subject is authorized to access a premise based on the two-dimensional visualizable code. When the subject is not authorized to access the premise based on the two-dimensional visualizable code, the second instructions cause the security gateway to: obtain second health readings of a subject via a second contactless sensor of the security gateway; interactively obtain second information from the subject in response to second prompts via a second user interface of the security gateway; measure second vital signs based on the second health readings of the subject; control the second user interface of the security gateway to present the second prompts, and interpret the second information interactively obtained from the subject in response to the second prompts. Access to the premise is conditional based on the second vital signs and the second information.

According to another aspect of the present disclosure, a security gateway for touch-free infectious disease screening includes a contactless sensor, a memory, and a processor. The contactless sensor is configured to obtain health readings of a subject. The memory stores instructions. The processor executes the instructions. When executed by the processor, the instructions cause the security gateway to measure vital signs based on the health readings of the subject, and determine whether to allow access to a premise based on the vital signs.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a communication device for touch-free infectious disease screening, in accordance with a representative embodiment.

FIG. 1B illustrates a security gateway for touch-free infectious disease screening, in accordance with a representative embodiment.

FIG. 1C illustrates an extraction arrangement using a camera for touch-free infectious disease screening, in accordance with a representative embodiment.

FIG. 1D illustrates a flow diagram for touch-free infectious disease screening, in accordance with a representative embodiment.

FIG. 2A illustrates a flow diagram for touch-free infectious disease screening, in accordance with a representative embodiment.

FIG. 2B illustrates a method for touch-free infectious disease screening, in accordance with a representative embodiment.

FIG. 3A illustrates another flow diagram for touch-free infectious disease screening, in accordance with a representative embodiment.

FIG. 3B illustrates another flow diagram for touch-free infectious disease screening, in accordance with a representative embodiment.

FIG. 4A illustrates a security gateway system for touch-free infectious disease screening, in accordance with a representative embodiment.

FIG. 4B illustrates elements used at a security gateway for touch-free infectious disease screening, in accordance with a representative embodiment.

FIG. 5 illustrates a communication device with user interfaces for touch-free infectious disease screening, in accordance with a representative embodiment.

FIG. 6 illustrates a computer system, on which a method for touch-free infectious disease screening is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, the infectious disease screening may be implemented in a manner that mitigates the disadvantages of temperature-only screening. The infection disease screening described herein includes one or more of the following characteristics:
Safe: does not contribute to disease spread
Autonomous: does not require operator
Effective: false negatives well below those of temperature-only screening
Large-scale: used in settings with high people throughput and deployed globally
Rapid: screening time <5 seconds; minimal impact on people throughput
Reliable: hard to cheat
Privacy safe: does not collect/display patient data
Evidence based: decision algorithm based on real data
Disease independent: can be used for outbreaks of various diseases
Setting independent: can be used at different types of setting
Airports, stadiums, office buildings, . . .
Easy to deploy: can rapidly deployed in case of a new outbreak

The teachings herein contemplate that a variety of the following elements may be used in multiple different combinations, including contactless vital signs measurements, questionnaires, face verification, movement tracking, use of AI to assess likelihood of disease. Questionnaires may be used to enter information that helps assess likelihood of infection. Face verification may be used to verifies that a subject submitting information remotely from a premise to be accessed (i.e., offsite) is the same person who shows up onsite at the premise. AI may be used to assess likelihood of disease based on all collected data.

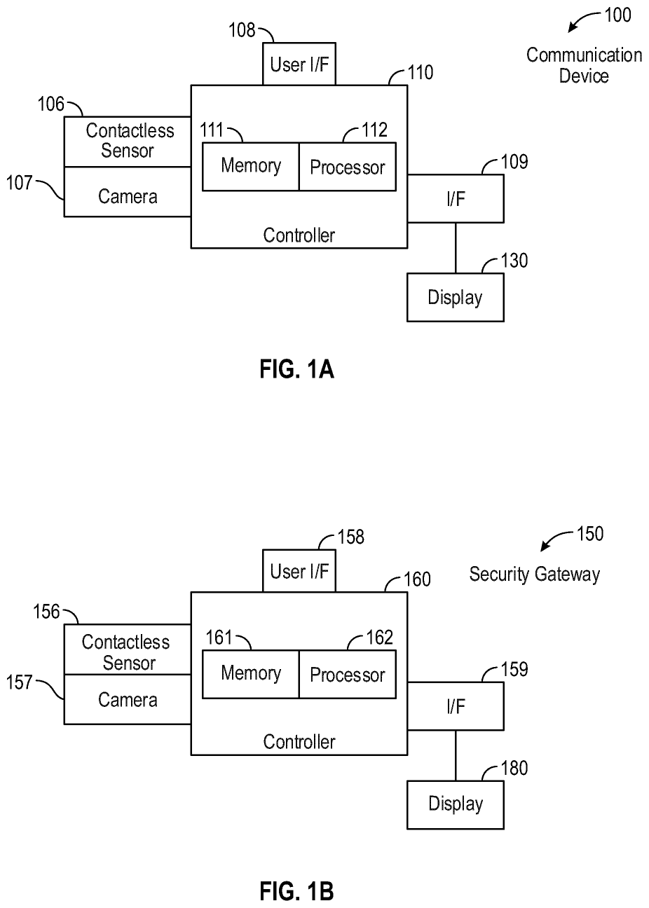

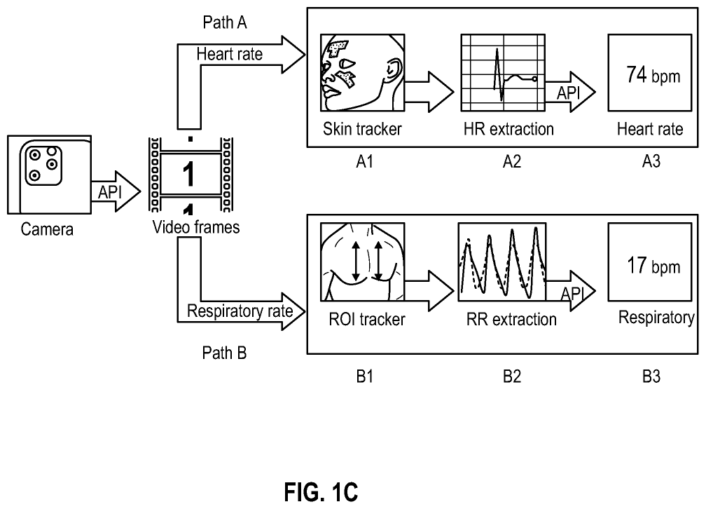

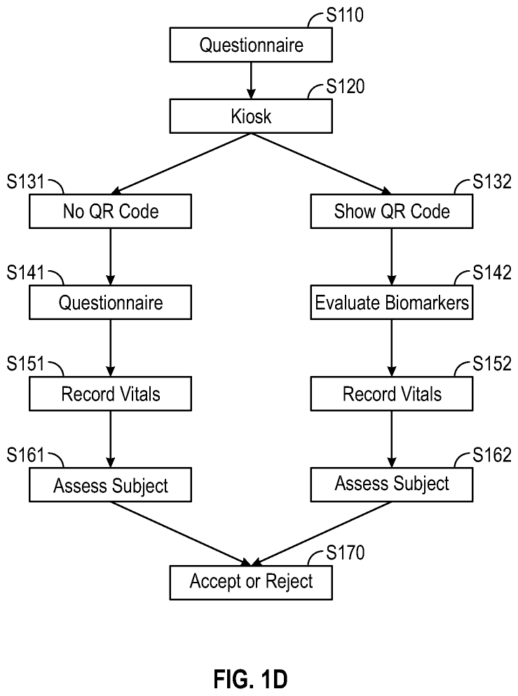

FIG. 1A illustrates a communication device 100 for touch-free infectious disease screening, in accordance with a representative embodiment.

The communication device 100 in FIG. 1A is a system for touch-free infectious disease screening via computer vision and includes components that may be provided together or that may be separable from one another. The communication device 100 includes a contactless sensor 106, a camera 107, a user interface 108, an interface 109, a controller 110 and a display 130. The controller 110 includes a memory 111 and a processor 112. The memory 111 stores instructions and the processor 112 executes the instructions. An example of the communication device 100 is a smartphone. The controller 110 may perform some of the operations described herein directly and may implement other operations described herein indirectly. That is, processes implemented by the controller 110 when the processor 112 executes instructions from the memory 111 may include steps not directly performed by the controller 110. The communication device 100 may be a smart phone which can be used to answer predetermined questions in a questionnaire and may also be used to take contactless vital signs measurement, and generate a quick response (QR) code or other equivalent two-dimensional visualizable code such as a bar code. In some embodiments, a code that is generated may be non-visualizable, such as a set of data (e.g., a byte or two bytes) that are limited to use by a near-field communication (NFC) module in the same manner as a two-dimensional visualizable code described herein. The same function may also be achieved by other means, such as encoded audio or another form of text or graphic which may be interpretable and understandable to a human gatekeeper. Any form of code described herein may be encrypted as a default except when retrieved by a user. The communication device 100 may also communicate with a smart watch or other wearable such as a fitness tracker that can take vital sign measurements, and incorporate data from the smart watch or other wearable into the analyses described herein.

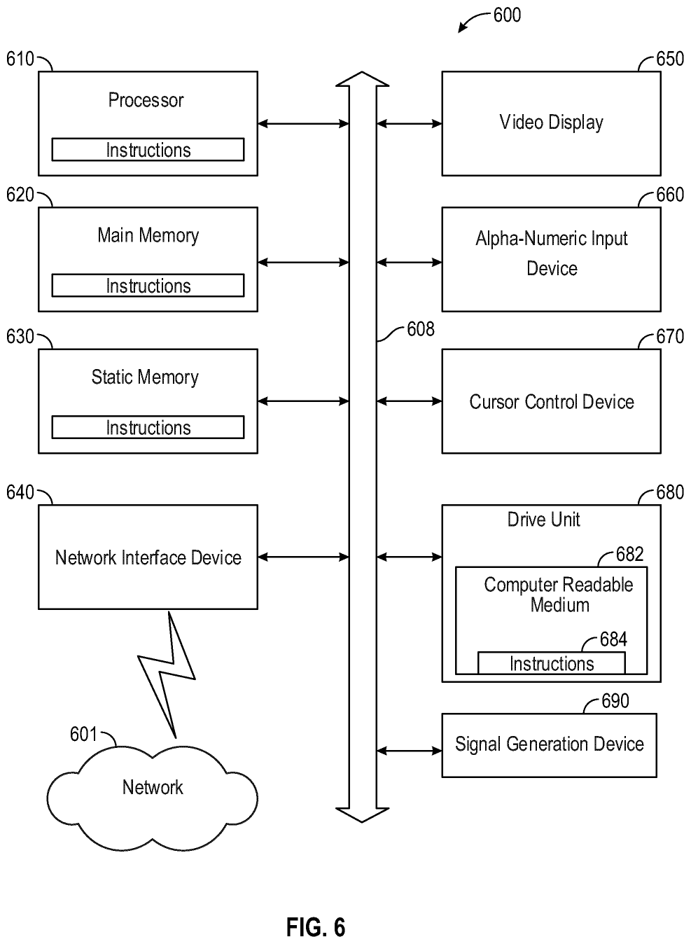

The communication device 100 is a computer for touch-free infectious disease screening via computer vision. The communication device 100 may be connected to one or more communication networks via interfaces. A computer that can be used to implement the communication device 100 is depicted in FIG. 6, though such a computer may include more or fewer elements than depicted in FIG. 6.

The contactless sensor 106 is configured to obtain health readings of a subject, and may be or include an infrared camera or thermometer. For example, health readings may be measured by obtaining video indicative of health readings and analyzing the video to extract vital signs. The camera 107 may be or include an RGB camera. The camera 107 may also be configured to obtain health readings of a subject, such as in the case of Ebola wherein eye color may reflect the presence of Ebola and which may be detected using an RGB camera. The user interface 108 may be configured to interactively obtain information from the subject in response to prompts. An example of the user interface 108 is a graphical user interface that displays prompts and virtual keys which can be used by the subject to respond to the prompts. Alternatively, the user interface 108 may be a combination of a graphical user interface that displays prompts and hard keys or soft keys which can be used by the subject to responds to the prompts. Alternatively, the user interface 108 may be or include a combination of a microphone and processor that capture voice commands and execute user voice recognition, with the voice dialogue optionally displayed on the display.

The processor 112 executes the instructions otherwise stored in the memory 111. When executed by the processor 112, the instructions cause the communication device 100 to: measure vital signs based on the health status of the subject; control the user interface to present the prompts, and interpret the information interactively obtained from the subject in response to the prompts; and encode results of measuring the vital signs and interpreting the information in a two-dimensional visualizable code. Access to a premise may conditioned on the two-dimensional visualizable code. An example of the two-dimensional visualizable code is a quick response (QR) code, though the teachings of the instant application are not limited to QR codes. QR codes (or equivalent) may be used to encode screening results and identifying information of a subject such as biometric input from an image of a face of the subject. For example, when a communication device 100 such as a smartphone is used to complete the questionnaire, submission of the questionnaire may require the user to allow the questionnaire application to use the camera 107 to record biometric identifiers of the user's face. Collection of the user's biometric identifiers may occur simultaneously with usage of the camera 107 to collect the user's vital sign measurements. The use of a QR code to convey health data and authorization to access a premise may help ensure privacy in that no communication of data over a network is involved, no vital signs are shown on the display 130, and neither the communication device 100 nor the security gateway 50 requires retention of any particular health data of the subject other than the QR code. The communication device 100 may encode biomarkers based on the biometric identifiers in the QR code.

The communication device 100 may also include interfaces to other components and devices. For example, the communication device 100 may be connectable to additional devices such as a display and user input devices such as a keyboard, and may include interfaces to the additional devices. Interfaces between devices and components described herein may include ports, disk drives, wireless antennas, or other types of receiver circuitry, as well as user interfaces such as a mouse, a keyboard, a microphone, a video camera, a touchscreen display, or other forms of interactive user interfaces.

The communication device 100 may also include a GPS-based location tracker. GPS data may be used to verify people have not been in hotspot areas. GPS data may automatically be checked by the program that executes the questionnaires for the communication device 100, or by the processor 112 executing instructions to process the data from the contactless sensor 106.

FIG. 1B illustrates a security gateway 150 for touch-free infectious disease screening, in accordance with a representative embodiment.

The security gateway 150 in FIG. 1B is a system for touch-free infectious disease screening via computer vision and includes components that may be provided together or that may be separable from one another. The security gateway 150 includes a contactless sensor 156, a camera 157, a user interface 158, an interface 159, a controller 160 and a display 180. The controller 160 includes a memory 161 and a processor 162. The memory 161 stores instructions and the processor 162 executes the instructions. An example of the security gateway 150 is a system that includes multiple instances of the components shown, wherein each instance is a tablet computer. The controller 160 may perform some of the operations described herein directly and may implement other operations described herein indirectly. That is, processes implemented by the controller 160 when the processor 162 executes instructions from the memory 161 may include steps not directly performed by the controller 160.

The security gateway 150 is a computer for touch-free infectious disease screening via computer vision. The security gateway 150 may be connected to one or more communication networks via interfaces. A computer that can be used to implement the security gateway 150 is depicted in FIG. 6, though such a computer may include more or fewer elements than depicted in FIG. 6.

The contactless sensor 156 is configured to obtain health readings of a subject, and may be or include an infrared camera or thermometer. The camera 157 may be or include an RGB camera. The user interface 158 may be configured to interactively obtain information from the subject in response to prompts. An example of the user interface 158 is a graphical user interface that displays prompts and virtual keys which can be used by the subject to respond to the prompts. Alternatively, the user interface 158 may be a combination of a graphical user interface that displays prompts and hard keys or soft keys which can be used by the subject to responds to the prompts. Also or alternatively, the user interface 158 may include a microphone or other voice interface which accepts voice input interpretable by a processor.

The processor 162 executes the instructions otherwise stored in the memory 161. When executed by the processor 162, the instructions cause the security gateway 150 to: measure vital signs based on the health readings of the subject; determine whether to allow access to a premise based on the vital signs.

The security gateway 150 may also include interfaces to other components and devices. For example, the security gateway 150 may be connectable to additional devices such as a display and user input devices such as a keyboard, and may include interfaces to the additional devices. Interfaces between devices and components described herein may include ports, disk drives, wireless antennas, or other types of receiver circuitry, as well as user interfaces such as a mouse, a keyboard, a microphone, a video camera, a touchscreen display, or other forms of interactive user interfaces.

If communication is allowed, and privacy-permitting, the security gateway 150 may receive pre-approval codes from the communication device 100 directly, such as over a communication network. In some embodiments, the pre-approval or other assessment results may be aggregated at a security gateway 150 or other centralized system to help track spread of a disease. The pre-approval codes may be sent over a communication network without requiring the pre-approval code to be displayed on the communication device 100 by the user at the security gateway 150.

An example of a contactless sensor 106, a contactless sensor 156 or other contactless mechanism for measuring vital signs, such as, e.g., heart rate, respiration rate, and/or blood oxygen saturation, is an optical measurement approach which uses remote photoplethysmography. An approach using remote photoplethysmography is described in the U.S. Provisional Patent Application No. 61/844,453, which is specifically incorporated by reference. (A copy of U.S. Provisional Patent Application No. 61/844,453 is attached.) Another example of a contactless sensor 106, a contactless sensor 156 or other contactless mechanism for measuring vital signs, including periodic vital signs such as, e.g., pulse, heart rate, and/or respiratory rate, is a remote photoplethysmographic analysis approach as described in commonly-owned U.S. Pat. No. 9,385,768, which is specifically incorporated by reference in its entirety. (A copy of U.S. Pat. No. 9,385,768 is attached.) Another example of a contactless sensor 106, a contactless sensor 156 or other contactless mechanism for measuring vital signs, including periodic vital signs such as, e.g., pulse, heart rate, and/or respiratory rate, through a remote photoplethysmographic analysis approach, is the approach described in the U.S. Pat. No. 8,649,562, which is herein incorporated by reference in its entirety. (A copy of U.S. Pat. No. 8,649,562 is attached.) Additionally, in some embodiments, audible physiological behavior that may be indicative of infection such as, e.g., coughing or sneezing, may also be detected to further support symptom detection through contactless mechanisms. As disclosed in the provisional patent application and patent applications incorporated herein by reference, contactless monitoring of vital signs and context may be captured via camera-based sensing software that detects heart and respiratory rate, wherein remote PPG (skin color changes) are used to determine heart rate, and the movement of chest and abdomen is analyzed to extract respiratory rate.

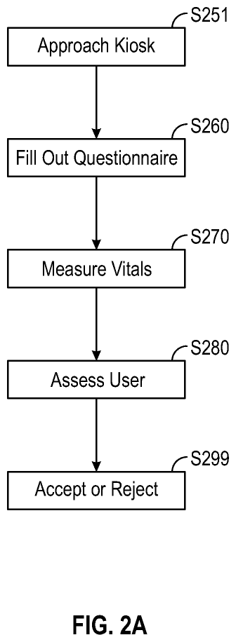

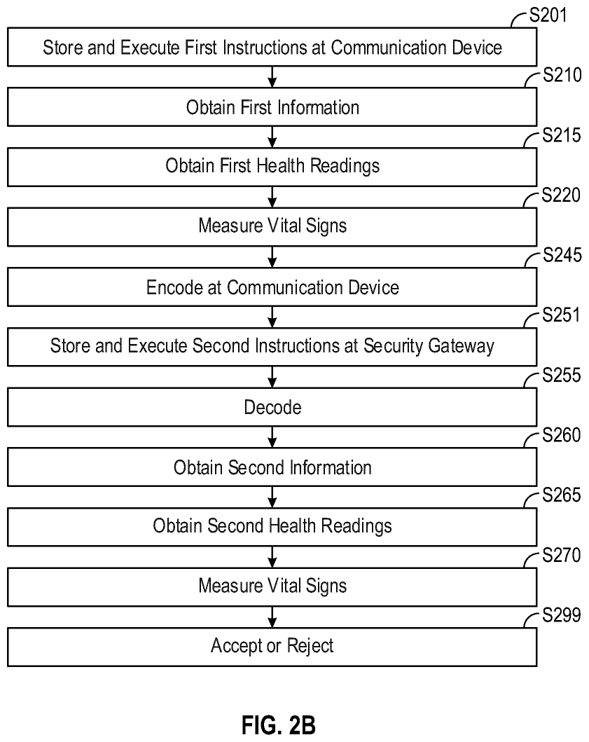

The use of the communication device 100 in FIG. 1A and the security gateway 150 in FIG. 1B eliminates any requirement for human-to-human screening in a screening process. When the communication device 100 and the security gateway 150 are used together, as described with respect to the embodiment of FIG. 2B below, reliability of the screening may be enhanced via, e.g., usage of pre-approval codes and/or biometric identifiers to increase the difficulty for an individual to bypass screening without being approved by the screening mechanisms. The use of one or both of the communication device 100 and/or the security gateway 150 for screening helps achieve comprehensive screening with high sensitivity and specificity while maintaining safety and an acceptable screening time, and without contributing to disease spread by, e.g., providing a contactless and/or autonomous screening approach.

FIG. 1C illustrates an extracting arrangement using a camera for touch-free infectious disease screening, in according with a representative embodiment.

In FIG. 1C, contactless monitoring of vital signs and context may be captured via camera-based sensing software that detects heart and respiratory rate. Remote PPG (skin color changes) are used to determine heart rate, and the movement of chest and abdomen is analyzed to extract respiratory rate. The camera in FIG. 1C may be the camera 107. Specifically, the camera 107 provides video frames which are used in path A for detecting a heart rate and in path B for detecting respiratory rate. Path A includes tracking skin at A1, extracting heartrate at A2 and outputting a heartrate via an application programming interface for display at A3. Path B includes tracking a region of interest (ROI) at B1, extracting a respiratory rate at B2, and generating and displaying the respiratory rate at B3.

Among the benefits offered by the approach in FIG. 1C is that it is (1) accurate, remote and unobtrusive, (2) provides simultaneous heart/breathing rate measurements, (3) is robust to motion and light variations, and (4) can be combined with facial recognition software

Measurements of health characteristics of a subject used herein may include pulse rate, respiration rate, SpO2, temperature and more, such as via a camera 107 or other contactless sensor(s) described herein. Some embodiments employ a camera 107 to measure pulse and respiratory rate to generate the health snapshot, and identify trends over time. Use of a camera 107 in this manner provides a health snapshot at points of entry and may be integrated with existing screening protocols to enhance systems beyond temperature checks. Additional contactless measurements may be integrated with measurements determined from the contactless sensor 106 or the contactless sensor 156. Such additional contactless measurements that may be implemented in the processing described herein may include: cough/sneeze detection from audio picked up by a microphone of the communication device 100 or the security gateway 150; cough/sneeze detection from video picked up by the camera 107 or the camera 157; cough/sneeze detection from a microphone and video camera; audio and video; shortness of breath detection from audio, video or even from another sensor such as a smartwatch or fitness tracker; and/or eye redness detection such as for Ebola from the camera 107 or the camera 157.

FIG. 1D illustrates a flow diagram for touch-free infectious disease screening.

FIG. 1D shows an example flow diagram of an example method, system, or computer-readable medium according to an example embodiment of the present disclosure. This example embodiment provides a screening solution comprising an offsite questionnaire used in coordination with onsite contactless screening. The site described herein may be an indoor and/or outdoor premise to be accessed upon successful completion of the screening.

When the example screening solution of FIG. 1D is implemented, individuals are notified of screening procedures prior to actual screening when they plan on going to a place (the “site”) that has implemented infectious disease screening. Individuals may then, offsite, fill out a questionnaire in S110 via a screening application or program on their smartphone or other suitable communication device. The questionnaire includes questions indicative of likelihood of disease. For example, with COVID-19, questions could ask questions such as, e.g., whether the user feels unwell, whether the user has recently traveled to any of listed outbreak regions, whether the user has experienced a fever in the last 24 hours, and/or whether the user is experiencing shortness of breath, repeated coughing, and so on. Additionally, completion of S110 may require contactless measurement of vitals, such as pulse rate, respiration rate, and/or blood oxygen saturation, via the camera 107 of the communication device 100. While measuring the vital signs, the application or program may also collect biometric identifiers (or, “biomarkers”) of the user's face, collected via the camera 107 while the screening application or program measures the person's vital signs. Measurement of vital signs such as pulse rate, respiration rate, and/or blood oxygen saturation may be conducted via the camera 107. Upon completion of the questionnaire and vitals measurement, the smartphone application may run an assessment algorithm, which can have two outcomes: (i) cleared, or (ii) rejected.

If a user is cleared, the smartphone application or program will generate a pre-approval code, which encodes the collected information, which can include responses to questions in the questionnaire, biomarkers of the user's face, and/or the user's vitals. The pre-approval code can be valid for only a pre-determined amount of time, such as, e.g., 2 hours. The pre-approval code may also encode biometric identifiers of the patient's face.

If a user is rejected, the smartphone application or program will recommend the user not go to the site that is screening, and can warn the user that they will be barred from entry to the site.

When arriving at the site that has implemented infectious disease screening, users approach and interact with a kiosk in S120. The kiosk may be, e.g., a tablet computer hanging on a wall and connected to a thermal camera or thermal sensor. Following S120, the user's interaction with the kiosk depends on whether the user has a pre-approval code to display to the kiosk.

If the user has a valid pre-approval QR code, then the user displays this code in S132. Then, in S142, the kiosk may take as inputs the biomarkers in the QR code and check the biomarkers in the QR code against biomarkers of the user measured at the kiosk using a camera. In S142, the kiosk may check the two sets of biomarkers to confirm that the user before the kiosk is the same individual who completed the questionnaire offsite in S110. In some embodiments, S142 is not performed, and in other embodiments S142 is optionally performed based on a dynamic determination at the kiosk if a question is raised as to the identity of the user. Subsequently, in S152, the kiosk utilizes a contactless mechanism to measure the user's vital signs. Then, at S162, the kiosk assesses the user's vital signs, questionnaire results, and biomarkers, and, depending on pre-determined criteria, either accepts or rejects them at S170. If the user is accepted, the user is permitted entry. If the user is rejected, the user is denied entry, and may be requested to leave the premises or to try again after waiting for a few minutes. This waiting period allows the user's vitals to settle and can thereby reduce the likelihood of a false positive due to elevated vitals caused by physical activity just prior to screening.

At S141, a user approaching the kiosk does not have a valid QR code, due to, e.g., not taking the offsite questionnaire or not possessing a smartphone. The user then, in S141, fills out a questionnaire, which may be similar or identical to the questionnaire of S110, onsite. Measurement or recording of biomarkers is unnecessary if the questionnaire is filled out at S141, as the user is before the kiosk at all times. The questionnaire responses may be submitted through a contactless measure, such as through voice or gesture commands recorded by the kiosk. Alternatively, the questionnaire may be filled out elsewhere, to, e.g., increase kiosk throughput. Filling out the questionnaire elsewhere may require kiosk operators to interact with the user themselves. Regardless of the location or process for filling out the questionnaire, in subsequent S151, the user's vital signs are measured. This can be done through a contactless mechanism. Next, at S161, the recorded questionnaire responses and vital sign measurements are compared against pre-determined criteria to either accept or reject the user. At S170, if the user is accepted, the user is permitted entry. If the user is rejected, the user is denied entry, and may be requested to leave the premises. The user may also be asked to try again in a few minutes, or be asked to be screened by an onsite healthcare professional, if available.

FIG. 2A illustrates a flow diagram for touch-free infectious disease screening, in accordance with a representative embodiment.

FIG. 2A shows an example flow diagram for an example method, system, or computer-readable medium according to some example embodiments of the present disclosure. The example of FIG. 2A provides an onsite screening solution comprising onsite contactless screening without usage of an offsite questionnaire. When the example screening solution of FIG. 2A is implemented, an offsite questionnaire is not used. Instead, the example screening solution of FIG. 2A utilizes an on-site questionnaire and contactless vital sign measurement for screening.

When arriving at the site that implements infectious disease screening, users approach and interact with a kiosk at S251. The kiosk may be or include, e.g., a tablet computer hanging on a wall and connected to a thermal camera. The kiosk may include multiple tablet computers that each provide the functionality attributed to the security gateways described herein. The user then, in S260, fills out a questionnaire onsite. Measurement or recording of biomarkers is unnecessary if the questionnaire is filled out at S260, as the user is before the kiosk at all times. The questionnaire responses may be submitted through a contactless measure, such as through voice or gesture commands recorded by the kiosk. Alternatively, the questionnaire may be filled out elsewhere, to, e.g., increase kiosk throughput. Filling out the questionnaire elsewhere may require kiosk operators to interact with the user themselves. Regardless of the location or process for filling out the questionnaire, in subsequent S270, the user's vital signs are measured. This can be done through a contactless mechanism. Next, at S280, the recorded questionnaire responses and vital sign measurements are compared against pre-determined criteria to either accept or reject the user. At S299, if the user is accepted, the user is permitted entry. If the user is rejected, the user is denied entry, and may be requested to leave the premises.

FIG. 2B illustrates a method for touch-free infectious disease screening, in accordance with a representative embodiment.

FIG. 2B is a comprehensive method that may be used when touch-free infectious disease screening is implemented at both a communication device 100 and a security gateway 150. To be clear, however, touch-free screening may be implemented based on subsets of the features in FIG. 2B solely implemented at a communication device 100 or a security gateway 150. In other words, not all of the features in FIG. 2B need to be performed in order to perform touch-free infectious disease screening as described herein.

At S201, the method of FIG. 2B includes storing and executing first instructions at a communication device. The communication device may be the communication device 100.

At S210, first information is obtained. The first information may be obtained at the communication device 100, and may be obtained via interactive prompts displayed via the display 130. The first information may be information obtained via a questionnaire, such as a predetermined script used to generate the interactive prompts and obtain and process the responses to the interactive prompts. An offsite questionnaire may be advantageous in that it may speed up onsite screening and/or provide additional vital signs measurement, which may reduce false positives caused by, e.g., skewed vitals caused by activity such as brisk walking prior to onsite vitals measurement. When a questionnaire is filled out offsite via an application or program on the communication device 100, the application or program may provide two outcomes to the user upon completion of the questionnaire and any measurement of the user's vital signs that the application or program may require. One outcome may be rejection, wherein the application or program may recommend the user not to go to the place that is screening and contact their healthcare provider, and may not provide a pre-approval code to the user. Another such outcome may be approval, wherein the application or program may generate a pre-approval code that encodes the user's collected information. At S215, first health readings are obtained. The first health readings may be obtained at the communication device 100, and may be obtained via the contactless sensor 106 sensing temperature readings of a subject using the communication device 100.

At S220, vital signs are measured. The vital signs may be measured by the processor 112 executing instructions to process the first health readings from the contactless sensor 106, such as by processing a time-series of first health readings.

At S245, the method of FIG. 2B includes encoding information at the communication device. The encoded information may be, may include, or may be based on the first information obtained at S210 and the vital signs measured at S245. The encoded information may also include a time-stamp to indicate the start of a period for which a health assessment is valid. The encoded information may also include results of the communication device 100 encoding biomarkers based on an image of a face of the subject at or immediately after the subject taking or completing a questionnaire. The communication device may encode biomarkers and may encode a timestamp for the time when the biomarkers are encoded. Any timestamp described herein may be used to mark the starting time for a time period in which the encoded information is valid, such as two hours after a subject is cleared to access a premise. The encoded information may be encoded in a two-dimensional visualizable code, such as a QR code. Use of a pre-approval code on the communication device 100 may prevent users from “cheating” the screening process. These users may be people who know that they are infected or likely to be infected, but nevertheless want to enter the site, and therefore have a healthy individual take measurements of their vital signs rather than themselves, i.e., the “cheating” user. In such embodiments, the pre-approval code may encode information regarding the user's biometric identifiers to prevent “cheating” the system. In some embodiments, usage of a pre-approval code which encodes data of a user's biometric identifiers may provide a screening solution that prevents cheating while also preserving the user's privacy. In those embodiments, no communication of data is involved, and therefore data regarding the user's biometric identifiers and vital sign measurements may never be transferred from the communication device 100. In further embodiments, vital sign measurements may not be shown on the communication device 100 except for as encoded in the pre-approval code.

At S251, the method of FIG. 2B includes storing and executing second instructions at a security gateway. The security gateway may be the security gateway 150, and the instructions may be stored at the security gateway 150 prior to S210 and executed later at S251.

At S255, the information encoded at S245 is decoded, such as by the security gateway 150 scanning the display 180 of the communication device 100, or the communication device 100 providing the encoded information directly to the security gateway 150 such as by Bluetooth or another short-range communication protocol. The camera 157 or another imaging device or component at the security gateway may be used for scanning the two-dimensional visualizable codes (e.g., QR codes) described herein. The security gateway 150 may decode the scanned or otherwise-received information from the communication device 100.

At S260, second information is obtained. The second information may be obtained at the security gateway 150, and may be obtained via interactive prompts displayed via the display 180. The second information may be information obtained via a questionnaire, such as a predetermined script used to generate the interactive prompts and obtain and process the responses to the interactive prompts.

A questionnaire that may be accessible on-site at a security gateway 150 may allow a subject to record responses via an electronic device, such as, e.g., a smartphone, tablet computer, or computer at the security gateway 150. Alternatively, an individual may record responses to the questionnaire through a contactless mechanism, such as, e.g., voice or by gestures. Contactless recording of responses may be facilitated through an automated solution that does not require an operator at the security gateway 150 for the screening technology. Contactless recording of responses may also or alternatively use automated language recognition or video analysis to identify, e.g., gestures or voice indicating responses to the questionnaire.

At S265, second health readings are obtained. The second health readings may be obtained at the security gateway 150, and may be obtained via the contactless sensor 156 sensing temperature readings of a subject present at the security gateway 150. Onsite systems at security gateways 150 may use separate systems for recording responses to a questionnaire at S260 and for measurement of vital signs at S265.

At S270, vital signs are measured. Measuring may include obtaining the video and/or any other readings from the contactless sensor 156, and deriving the vital signs from the video. The vital signs may be measured by the processor 162 executing instructions to process the second health readings from the contactless sensor 156, such as by processing a time-series of second health readings.

At S299, a decision is made to accept or reject the subject from access to the premise. The decision at S299 may be made by the processor 162, and may be based on an analysis of the second information received at S260 and the vital signs measured at S270.

To implement touch-free infectious disease screening solely at a communication device 100, the features from S201 to S220 may be performed along with any other necessary or appropriate functions. To implement touch-free infection screening solely at a security gateway 150, the features from S260 to S299 may be performed along with any other necessary or appropriate functions. However, in an integrated example where some aspects of touch-free infectious disease screening are implemented away from a premise to be accessed, the screening may be performed both remotely from the security gateway 150 and then at the security gateway 150, such as by using the encoded two-dimensional visualizable codes described herein. Using the features of FIG. 2B and other embodiments herein, enhanced venue safety protocols are provided via one or more real-time snapshot(s) of temperature, pulse and respiratory rate. Embodiments based on some or all of the features in FIG. 2B may reduce the likelihood of false negatives by, e.g., collecting data, which may be self-reported or measured on-site, on individuals regarding symptoms beyond only fever.

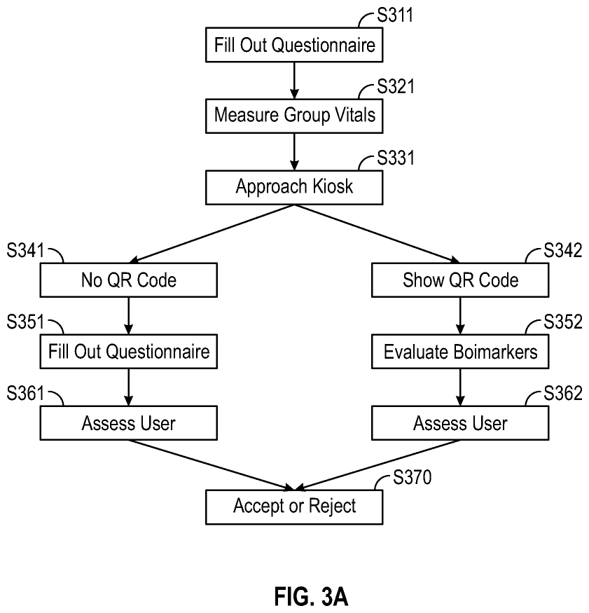

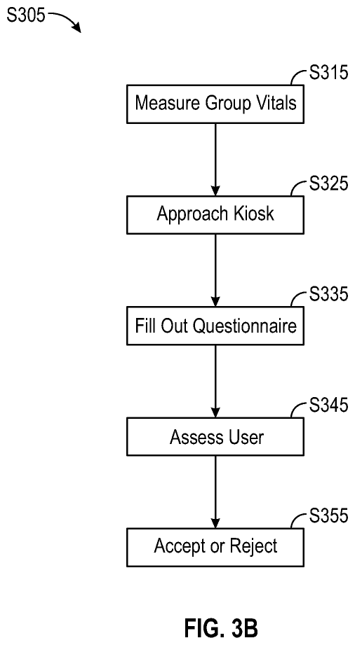

FIGS. 3A and 3B show example flow diagrams 300 and 305 of an example method, system, or computer-readable mediums according to two example embodiments of the present disclosure. These example embodiments provide onsite screening solutions which aim for high-throughput screening with no offsite vitals measured. An offsite questionnaire may be involved, as in example flow diagram 300, or may not be used, as in example flow diagram 305.

FIG. 3A illustrates another flow diagram for touch-free infectious disease screening, in accordance with some representative embodiments.

FIG. 3A shows example flow diagram 300 of an example method, system, or computer-readable medium according to an example embodiment of the present disclosure. When this example screening solution is implemented, individuals are notified of screening procedures prior to actual screening when they plan on going to a place (the “site”) that has implemented infectious disease screening. Individuals may then, offsite, fill out a questionnaire at S311 via a screening application or program on their smartphone. The questionnaire includes questions indicative of likelihood of disease. For example, with COVID-19, questions could ask questions such as, e.g., whether the user feels unwell, whether the user has recently traveled to any of listed outbreak regions, whether the user has experienced a fever in the last 24 hours, and/or whether the user is experiencing shortness of breath. Additionally, completion of S311 can require contactless measurement of vitals, such as pulse rate, respiration rate, and/or blood oxygen saturation, via the user's smartphone camera. While measuring the user's vitals, the application or program can also collect biometric identifiers (or, “biomarkers”) of the user's face, collected via the camera 107 while the screening application or program measured the person's vitals.

Upon completion of the questionnaire and vitals measurement, the smartphone application or program runs an assessment algorithm, which can have two outcomes: (i) cleared, or (ii) rejected.

If a user is cleared, the smartphone application or program will generate a pre-approval code, which encodes the collected information, which can include responses to questions in questionnaire 311, biomarkers of the user's face, and/or the user's vitals. The pre-approval code may be valid for only a pre-determined amount of time, such as, e.g., 2 hours.

If a user is rejected, the smartphone application or program will recommend the user not go to the site that is screening, and can warn the user that they will be barred from entry to the site.

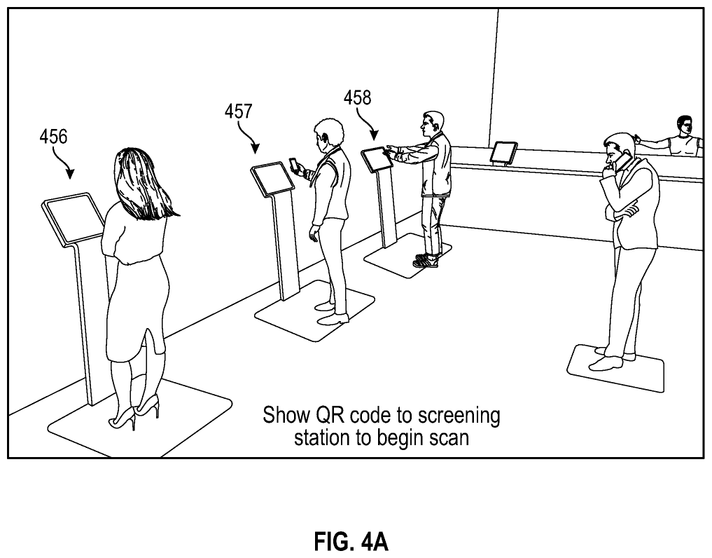

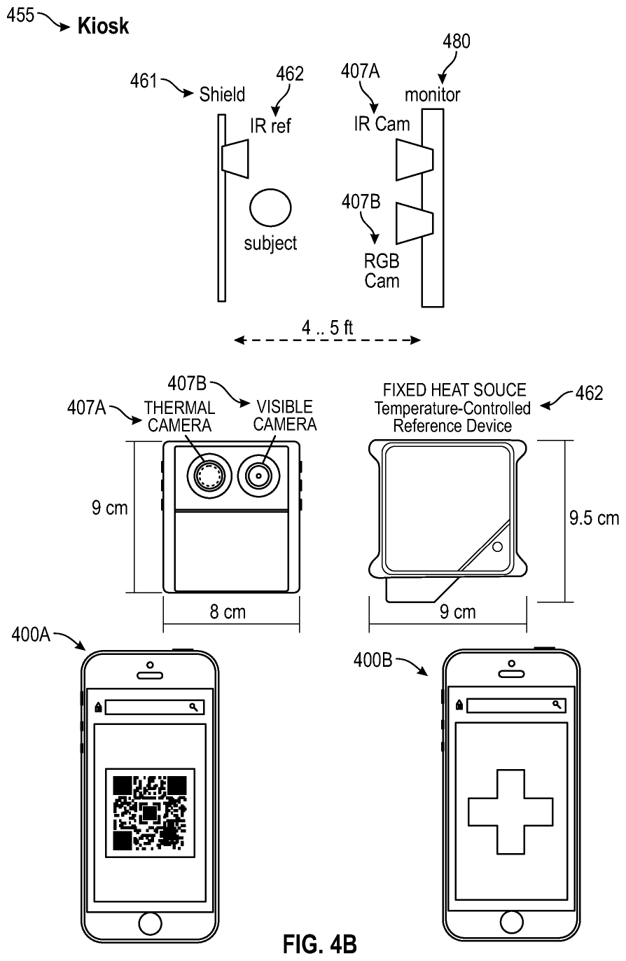

When arriving at the site that has implemented infectious disease screening, users wait in line to be individually screened by a kiosk in S321. Users waiting in line for screening at a security gateway system are shown in FIG. 4A. An example of a configuration for a kiosk is shown in FIG. 4B. In FIG. 4A, a temperature reference is not used, whereas in FIG. 4B, a temperature reference is used. FIG. 4A achieves higher screening throughput by making multiple kiosks available in parallel. The kiosk measures vital signs of multiple subjects simultaneously. Different kiosks with different cameras may be used for the different measurements. During S321, the vital signs of multiple users are measured in parallel, and cameras may track each user from the place in line where the measurement is initiated to where the measured user begins interfacing with the respective kiosk used by the user at S331, decreasing the time necessary for each user to spend in screening at the kiosk used by the user. Using the teachings herein, vital signs of one or multiple individuals may be measured without medical personnel actually coming into contact with the individual or individuals, and this may improve safety. As set forth above, a security gateway system may be configured to obtain images of subjects, screen the subjects using health readings of the subjects obtained from a contactless sensor, and track movements of the subjects using the images.

The users approach and interact with a kiosk in S331. The kiosk may be, e.g., a tablet computer hanging on a wall and connected to a thermal camera. Following S331, the user's interaction with the kiosk depends on whether the user has a pre-approval code to display to the kiosk.

If the user has a valid QR code, then the user displays the QR code in S342. Then, in S352, the kiosk takes as inputs the biomarkers in the QR code and checks them against biomarkers and measures biomarkers of the user, using a camera. In S352, the kiosk checks the two sets of biomarkers to confirm that the user before the kiosk is the same individual who completed the questionnaire offsite in S311. Then, in S362, the kiosk assesses the user's vital signs, questionnaire results, and biomarkers, and, depending on pre-determined criteria, either accepts or rejects them in S370. If the user is accepted, the user is permitted entry. If the user is rejected, the user is denied entry, and may be requested to leave the premises.

In S341, a user approaching the kiosk does not have a valid QR code, due to, e.g., not taking the offsite questionnaire or not possessing a smartphone. The user then, in S351, fills out a questionnaire, which may be similar or identical to the questionnaire of S311, onsite. Measurement or recording of biomarkers is unnecessary if the questionnaire is filled out at S341, as the user is before the kiosk at all times. The questionnaire responses may be submitted through a contactless measure, such as through voice or gesture commands recorded by the kiosk. Alternatively, the questionnaire may be filled out elsewhere, to, e.g., increase kiosk throughput. Filling out the questionnaire elsewhere may require kiosk operators to interact with the user themselves, though this may be avoided using an interface that is configured to accept and interpret voice input and/or gesture input. Next, at S361, the recorded questionnaire responses and vital sign measurements are compared against pre-determined criteria to either accept or reject the user. At S370, if the user is accepted, the user is permitted entry. If the user is rejected, the user is denied entry, and may be requested to leave the premises.

FIG. 3B illustrates another flow diagram for touch-free infectious disease screening, in accordance with a representative embodiment.

FIG. 3B shows example flow diagram 305 of an example method, system, or computer-readable medium according to an example embodiment of the present disclosure. This example embodiment provides an onsite screening solution comprising onsite contactless screening without usage of an offsite questionnaire. When this example screening solution is implemented, an offsite questionnaire is not used. Instead, the example screening solution utilizes an on-site questionnaire and contactless vital sign measurement of multiple users in parallel to screen users.

When arriving at the site that has implemented infectious disease screening, users wait in line to be individually screened by a kiosk at S315. While waiting in line, the kiosk performs multiple vital sign measurements on multiple people at once. Different cameras may be used for different measurements. During S315, the vital signs of multiple users are measured in parallel, while the camera(s) track(s) each user from the place in line where the measurement is initiated to where the measured user begins interfacing with the kiosk at S325, decreasing the time necessary for each user to spend in screening at the kiosk.

The users approach and interact with a kiosk at S325. The kiosk may be, e.g., a tablet computer hanging on a wall and connected to a thermal camera. Following S325, the user's interaction with the kiosk depends on whether the user has a pre-approval code to display to the kiosk.

The user then, at S335, fills out a questionnaire onsite. Measurement or recording of biomarkers is unnecessary if the questionnaire is filled out at S335, as the user was tracked by a camera system. The questionnaire responses may be submitted through a contactless measure, such as through voice or gesture commands recorded by the kiosk. Filling out the questionnaire elsewhere may require kiosk operators to interact with the user themselves, though this may be avoided using an interface that is configured to accept and interpret voice input and/or gesture input. Next, at S345, the recorded questionnaire responses and vital sign measurements are compared against pre-determined criteria to either accept or reject the user. At S355, if the user is accepted, the user is permitted entry. If the user is rejected, the user is denied entry, and may be requested to leave the premises.

Multiple people may be measured in parallel since pulse rate measurement takes time, such as 20 seconds per subject. If the system passes a person every 5 seconds, the first 20/5=4 people in line may need to be measured in parallel. This way, when a person reaches the security gateway 150, his/her vitals are known, enabling the system to decide on allowing or denying entry. If an offsite questionnaire is involved, the QR pre-approval code may be scanned at the security gateway 150. The security gateway 150 tracks each person from the place in line where measurement is initiated to the security gateway 150, so as to have the right measurements available for each person at the security gateway 150.

FIG. 4A illustrates a security gateway system for touch-free infectious disease screening, in accordance with a representative embodiment.

In FIG. 4A, three different systems include a first system 456, a second system 457 and a third system 458. Each of the three systems at the security gateway system in FIG. 4A are shown to include a tablet computer which includes functionality for interactively obtaining information via a questionnaire, reading and processing a two-dimensional visualizable code from a user communication device, and sensing health characteristics of a user in order to measure vital signs. Privacy when answering a questionnaire onsite may be improved by keeping having people wait some distance from the system they will use until it is their turn.

FIG. 4B illustrates elements used at a security gateway for touch-free infectious disease screening, in accordance with a representative embodiment.

In FIG. 4B, a kiosk 455 includes a shield 461, an infrared reference 462 (IR reference), an infrared camera 407A, a monitor 480, and an RGB camera 407B. The layout of the infrared camera 470A and the RGB camera 407B is shown in the image on the middle left, and the layout of the infrared reference 462 is shown on the middle right. On the bottom left, FIG. 4B shows an example user interface 400A for a communication device 100, such as a display of a QR code. On the bottom right, FIG. 4B shows an example user interface 400B, such as an indication that a QR code was accepted so the subject is enabled to access a premise.

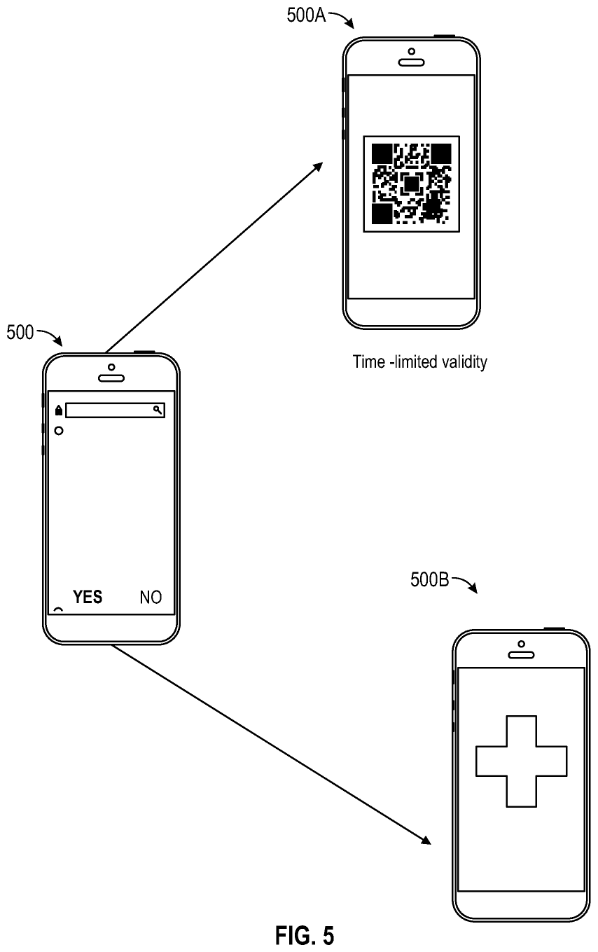

FIG. 5 illustrates a communication device with user interfaces for touch-free infectious disease screening, in accordance with a representative embodiment.

In FIG. 5, a communication device 500 is shown on the middle left. On the upper part of FIG. 5, a user interface 500A is shown with a QR code designated with a time-limited validity. For example, when the user passes screening at the communication device 500, the QR code on the user interface 500A may be issued for a limited period of time, such as 2 hours. When the user displays the QR code at the security gateway 150, the user is enabled to access a premise as indicated by the large positive sign on the user interface 500B.

FIG. 6 illustrates a computer system, on which a method for touch-free infectious disease screening is implemented, in accordance with another representative embodiment.

Referring to FIG. 6, the computer system 600 includes a set of software instructions that can be executed to cause the computer system 600 to perform any of the methods or computer-based functions disclosed herein. The computer system 600 may operate as a standalone device or may be connected, e.g., using a network 601, to other computer systems or peripheral devices. In embodiments, a computer system 600 performs logical processing based on digital signals received via an analog-to-digital converter.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as a communication device, a security gateway, a tablet computer at a security gateway, a control system for a security gateway, or another type of computer that includes the controller 110 in FIG. 1A or the controller 160 in FIG. 1B, including a stationary computer, a mobile computer, a personal computer (PC), a laptop computer, a tablet computer, or any other machine capable of executing a set of software instructions (sequential or otherwise) that specify actions to be taken by that machine. The computer system 600 can be incorporated as or in a device that in turn is in an integrated system that includes additional devices. In an embodiment, the computer system 600 can be implemented using electronic devices that provide voice, video or data communication. Further, while the computer system 600 is illustrated in the singular, the term “system” shall also be taken to include any collection of systems or sub-systems that individually or jointly execute a set, or multiple sets, of software instructions to perform one or more computer functions.

As illustrated in FIG. 6, the computer system 600 includes a processor 610. The processor 610 may be considered a representative example of the processor 112 of the controller 110 in FIG. 1A and of the processor 162 of the controller 160 in FIG. 1B and executes instructions to implement some or all aspects of methods and processes described herein. The processor 610 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 610 is an article of manufacture and/or a machine component. The processor 610 is configured to execute software instructions to perform functions as described in the various embodiments herein. The processor 610 may be a general-purpose processor or may be part of an application specific integrated circuit (ASIC). The processor 610 may also be a microprocessor, a microcomputer, a processor chip, a controller, a microcontroller, a digital signal processor (DSP), a state machine, or a programmable logic device. The processor 610 may also be a logical circuit, including a programmable gate array (PGA), such as a field programmable gate array (FPGA), or another type of circuit that includes discrete gate and/or transistor logic. The processor 610 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 600 further includes a main memory 620 and a static memory 630, where memories in the computer system 600 communicate with each other and the processor 610 via a bus 608. Either or both of the main memory 620 and the static memory 630 may be considered representative examples of the memory 111 of the controller 110 in FIG. 1A and of the memory 161 of the controller in FIG. 1B, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 620 and the static memory 630 are articles of manufacture and/or machine components. The main memory 620 and the static memory 630 are computer-readable mediums from which data and executable software instructions can be read by a computer (e.g., the processor 610). Each of the main memory 620 and the static memory 630 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, Blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 600 further includes a video display unit 650, such as a liquid crystal display (LCD), an organic light emitting diode (OLED), a flat panel display, a solid-state display, or a cathode ray tube (CRT), for example. Additionally, the computer system 600 includes an input device 660, such as a keyboard/virtual keyboard or touch-sensitive input screen or speech input (e.g., microphone) with speech recognition, and a cursor control device 670, such as a mouse or touch-sensitive input screen or pad. The computer system 600 also optionally includes a disk drive unit 680, a signal generation device 690, such as a speaker or remote control, and/or a network interface device 640. The computer system 600 may also include a speaker for audio output when a microphone is used to accept audio input.

In an embodiment, as depicted in FIG. 6, the disk drive unit 680 includes a computer-readable medium 682 in which one or more sets of software instructions 684 (software) are embedded. The sets of software instructions 684 are read from the computer-readable medium 682 to be executed by the processor 610. Further, the software instructions 684, when executed by the processor 610, perform one or more steps of the methods and processes as described herein. In an embodiment, the software instructions 684 reside all or in part within the main memory 620, the static memory 630 and/or the processor 610 during execution by the computer system 600. Further, the computer-readable medium 682 may include software instructions 684 or receive and execute software instructions 684 responsive to a propagated signal, so that a device connected to a network 601 communicates voice, video or data over the network 601. The software instructions 684 may be transmitted or received over the network 601 via the network interface device 640.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although touch-free infectious disease screening has been described with reference to several exemplary embodiments, it is understood that the words that have been used are words of description and illustration, rather than words of limitation. Changes may be made within the purview of the appended claims, as presently stated and as amended, without departing from the scope and spirit of touch-free infectious disease screening in its aspects. Although touch-free infectious disease screening has been described with reference to particular means, materials and embodiments, touch-free infectious disease screening is not intended to be limited to the particulars disclosed; rather touch-free infectious disease screening extends to all functionally equivalent structures, methods, and uses such as are within the scope of the appended claims. As one example, the teachings herein are applicable independent of any particular disease such as Covid-19, and this is due in some part to the use of data from multiple different categories of vital signs and/or potential symptoms. The use of the questionnaire and the use of contactless measurements may be varied and tailored for different diseases. The contactless measurements may be flexible in terms of what can be done via cameras and/or microphones in both the communication device 100 and the security gateway 150, so that measurements can be tailored to the symptoms of any particular disease.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description. The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A communication device for touch-free infectious disease screening, comprising:
a memory that stores instructions;
a contactless sensor configured to obtain health readings of a subject;
a user interface configured to interactively obtain information from the subject in response to prompts; and
a processor that executes the instructions, wherein, when executed by the processor, the instructions cause the communication device to:
measure vital signs based on the health readings of the subject;
control the user interface to present the prompts, and interpret the information interactively obtained from the subject in response to the prompts; and
encode results of measuring the vital signs and interpreting the information in a two-dimensional visualizable code, wherein, access to a premise is conditioned on the two-dimensional visualizable code.

2. The communication device of claim 1, wherein the vital signs are measured and the information is interactively obtained via the communication device when the communication device is remote from the premise.

3. The communication device of claim 1, wherein, when executed by the processor, the instructions further cause the communication device to:
analyze the vital signs and interpret the information to diagnose whether the subject is infected; and
encode whether the subject is infected in the two-dimensional visualizable code.

4. The communication device of claim 3, further comprising:
a camera, wherein, when the subject is not infected, the communication device is configured to obtain an image of the subject as a biometric input via the camera, and
wherein, when executed by the processor, the instructions further cause the communication device to:
encode biomarkers of the subject from the image along with results of analyzing the vital signs and interpreting the information in the two-dimensional visualizable code.

5. The communication device of claim 4, wherein, when executed by the processor, the instructions further cause the communication device to:
encode a timestamp of when the image of the subject is obtained in the two-dimensional visualizable code, wherein the timestamp is used as a starting time for a time-limit for using the results of analyzing the vital signs and interpreting the information.

6. The communication device of claim 1, wherein the premise screens subjects including the subject using two-dimensional visualizable codes including the two-dimensional visualizable code by scanning the two-dimensional visualizable codes.

7. The communication device of claim 1, wherein the information interactively obtained from the subject in response to prompts is interactively obtained via a questionnaire comprising predetermined questions.

8. A method for touch-free infectious disease screening, the method comprising:
storing first instructions in a first memory of a communication device;
obtaining first health readings of a subject via a first contactless sensor of the communication device;
interactively obtaining first information from the subject in response to first prompts via a first user interface of the communication device; and
executing the first instructions by a first processor of the communication device;
storing second instructions in a second memory of a security gateway; and
executing the second instructions by a second processor of the security gateway,
wherein, when executed by the first processor of the communication device, the first instructions cause the communication device to:
measure first vital signs based on the first health readings of the subject;
control the first user interface of the communication device to present the first prompts, and interpret the first information interactively obtained from the subject in response to the first prompts; and
encode first results of measuring the first vital signs and of interpreting the first information in a two-dimensional visualizable code;
wherein, when executed by the second processor of the security gateway, the second instructions cause the security gateway to:
scan the first user interface of the communication device to obtain the two-dimensional visualizable code;
decode the two-dimensional visualizable code;
determine whether the subject is authorized to access a premise based on the two-dimensional visualizable code; and
when the subject is not authorized to access the premise based on the two-dimensional visualizable code:
obtain second health readings of a subject via a second contactless sensor of the security gateway;
interactively obtain second information from the subject in response to second prompts via a second user interface of the security gateway;
measure second vital signs based on the second health readings of the subject; and
control the second user interface of the security gateway to present the second prompts, and interpret the second information interactively obtained from the subject in response to the second prompts, and
wherein, access to the premise is conditional based on the second vital signs and the second information.

9. A security gateway for touch-free infectious disease screening, comprising:
a contactless sensor configured to obtain health readings of a subject;
a memory that stores instructions; and
a processor that executes the instructions,
wherein, when executed by the processor, the instructions cause the security gateway to:
measure vital signs based on the health readings of the subject; and determine whether to allow access to a premise based on the vital signs.

10. The security gateway of claim 9, further comprising:
a user interface configured to interactively obtain information from the subject in response to prompts,
wherein, when executed by the processor, the instructions cause the security gateway to:
control the user interface to present the prompts, and interpret the information interactively obtained from the subject in response to the prompts, wherein the information interactively obtained from the subject in response to prompts is interactively obtained via a questionnaire comprising predetermined questions, and
wherein, whether to allow access to the premise is determined further based on the information interactively obtained from the subject.

11. The security gateway of claim 9, wherein, when executed by the processor, the instructions cause the security gateway further to:
decode results of a communication device measuring the vital signs in a two-dimensional visualizable code, and
determine that the two-dimensional visualizable code does not authorize access to the premise.

12. The security gateway of claim 9,
wherein, when executed by the processor, the instructions further cause the security gateway to:
analyze the vital signs to diagnose whether the subject is infected

13. The security gateway of claim 9, further comprising:
a camera, wherein the security gateway is configured to obtain an image of the subject as a biometric input via the camera, and
wherein, when executed by the processor, the instructions further cause the security gateway to:
obtain first biomarkers of the subject from an image taken by a communication device of the subject;
obtain second biomarkers of the subject from the image taken by the security gateway, and
compare the first biomarkers to the second biomarkers.

14. The security gateway of claim 13, wherein, when executed by the processor, the instructions further cause the security gateway to:
record a timestamp of when the image of the subject is obtained by the security gateway.

15. The security gateway of claim 9, further comprising:
a camera, wherein the security gateway is configured to obtain images of subjects including the subject,
wherein, when executed by the processor, the instructions further cause the security gateway to:
screen subjects including the subject using health readings of the subjects obtained from the contactless sensor, and
track movements of the subjects including the subject using the images.

16. The security gateway of claim 9, wherein the contactless sensor comprises at least one of: in infrared thermometer, a thermal camera, and a remote optical sensor.

17. The security gateway of claim 9, wherein the contactless sensor comprises an RGB camera.

18. The security gateway of claim 9, wherein the processor further encodes results of measuring the vital signs and interpreting the information in a two-dimensional visualizable code, and wherein determining whether to allow access to a premise is conditioned on the two-dimensional visualizable code.

19. The security gateway of claim 14, wherein the timestamp is used as a starting time for a time-limit for using the results of analyzing the vital signs and interpreting the information.

20. The security gateway of claim 16, wherein the measured vital sign comprises at least one of: pulse rate, respiration rate, SpO2, and temperature.
